(19) United States
(12) Patent Application Publication
(10) Pub. No.: US 2009/0110618 A1
(43) Pub. Date: Apr. 30, 2009

(54) SINK DRAIN DEODORIZER STICK

(76) Inventor: Thomas K. Dombrowski, Elk Grove Village, IL (US)

Correspondence Address: CLAUSEN MILLER, P.C, SUITE 1600, 10S. LASALLE STREET, CHICAGO, IL, 60603, US

(21) Appl. No.: 11/926,384

(22) Filed: Oct. 29, 2007

(51) Int. Cl. A61L 2/23

(52) U.S. Cl. 422/291

(57) ABSTRACT

A solid chemical stick for treating a kitchen sink drain pipe. The solid chemical stick has dimensions that enable it to fit through a drain guard but prevent the stick from moving beyond the drain trap. The stick diminishes in size as it dissolves until it is swept away by drain water passing through the drain pipe.

### BACKGROUND

1. Field of the Invention

This patent relates to a chemical deodorizer, sanitizer and/or freshener for a sink drain. More specifically, this patent relates to a solid chemical stick that nests in a sink drain trap and slowly dissolves to deodorize, sanitize and/or freshen the drain.

2. Description of the Related Art

Kitchen sink drains come equipped with traps designed to prevent gases from backing up into the kitchen. Water settling in the trap prevents the gases from passing upstream through the drain and into the kitchen. However, these gases can contaminate or otherwise sour the water in the trap, and the resulting unpleasant odor can disseminate back into the kitchen. Food and other particles trapped in the drain also can cause sink odors.

Numerous methods of dealing with this problem have been proposed, perhaps the most common being to pour baking soda (sodium bicarbonate) down the drain. While this method may provide a temporary solution to a drain odor problem, the baking soda is quickly washed away once water is introduced into the drain.

Other methods of deodorizing and or sanitizing sink drains have been proposed. For example, Gangloff U.S. Pat. No. 2,542,367 describes a device that fits in the trap of a sink drain and can be heated as needed to clean the drain trap. The heating effect can also sanitize the drain by killing organisms.

Roth U.S. Pat. No. 4,910,808 describes a device that dispenses a flowable deodorizing chemical from a pressurized container into the inside of a grinder type garbage disposal system.

Richter U.S. Pat. No. 6,197,321 describes a solid granular chemical drain treatment product that fits above a sink drain. The chemical slowly dissolves under contact with drain water.

Scuilla U.S. Pat. No. 6,558,538 describes a rod-shaped device that can be dropped down into the area within a U-shaped sink trap to sanitize the drain. The device is a porous container which holds a chemical deodorizer that can slowly dissolve. The device is held in place by a tether.

All of these methods involve complicated solutions to the problem of drain odors. Thus there remains a need for a simple, effective way to deodorize and/or sanitize a sink drain. The present invention is intended to meet that need by providing a solid chemical stick that can be dropped down a sink drain and then lodged in the typically U-shaped trap to allow the solid chemical deodorizing agent to slowly dissolve, thereby deodorizing, sanitizing and or freshening the drain.

Further and additional objects will appear from the description, accompanying drawings, and appended claims.

### SUMMARY OF THE INVENTION

The present invention is a solid chemical stick for treating a kitchen or industrial sink drain pipe. The sink itself comprises a basin and a drain guard installed in the basin. The drain pipe is connected to the drain guard and includes a trap which is typically U-shaped. The solid chemical stick has dimensions that enable it to fit through the drain guard but that prevent the stick from moving beyond the trap. The stick diminishes in size as it dissolves until it can be swept away by drain water passing through the drain pipe.

### THE DRAWINGS

FIG. 1 is a perspective view of a sink drain shown in partial cutaway to show the invention in use.

FIG. 2 is a perspective view of a drain guard.

### DETAILED DESCRIPTION OF THE INVENTION

While this invention may be embodied in many forms, there is shown in the drawings and will herein be described in detail one or more embodiments with the understanding that this disclosure is to be considered an exemplification of the principles of the invention and is not intended to limit the invention to the illustrated embodiments.

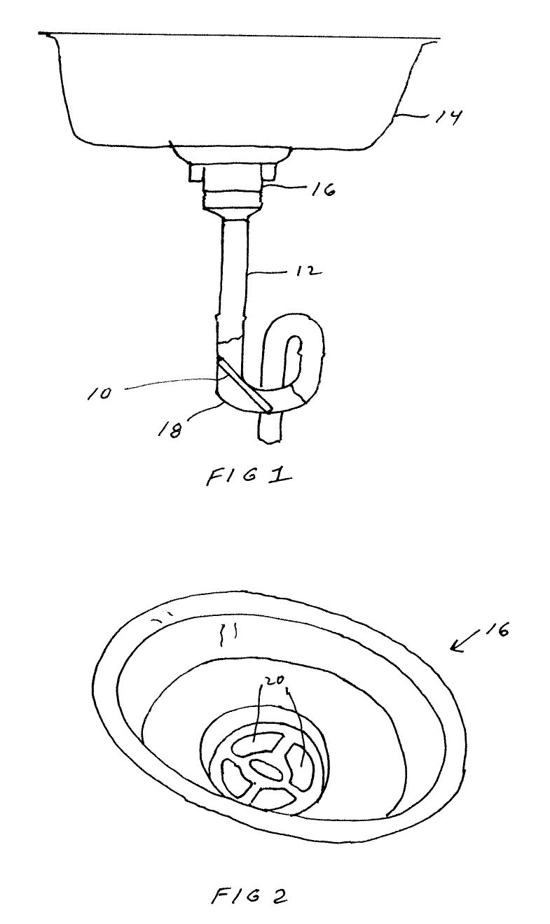

Turning to the drawings, there is shown in FIG. 1 a perspective view of a sink drain, shown in partial cutaway to show one embodiment of the invention in use. The invention is a solid chemical stick 10 used for treating a kitchen or industrial sink drain pipe 12. The sink itself comprises a basin 14 and a drain guard 16 (shown in greater detail in FIG. 2) installed in the basin 14. The drain pipe 12 is connected to the drain guard 16 and includes a typically U- or P-shaped trap 18, shown in cutaway view in FIG. 1. In an important aspect of the invention, the solid chemical stick 10 has dimensions that enable it to fit through the drain guard 16 but that prevent the stick 10 from moving beyond the trap 18. The stick 10 diminishes in size as it dissolves until it can be swept away by drain water passing through the drain pipe 12.

To use the invention, the user drops a stick 10 down through openings 20 in the drain guard 16. The stick 10 becomes lodged in the trap 18, and will remain there, freshening the drain, until enough of the stick dissolves to enable it to pass through and beyond the trap 18.

Although the invention has been termed a “stick”, the invention can have any dimensions as long as they are small enough to enable the stick to pass through the openings 20 in the drain guard 16 yet large enough to cause the stick to become lodged in the trap 18, at least until the stick partially dissolves. Suggested dimensions for the stick 10 are a minimum of one sixteenth inches in width or diameter and a minimum of about four and one half inches in length. Of course, the acceptable dimensions are a function of the size of the drain guard openings 20 and the trap 18.

The illustrated stick 10 is cylindrical and has a circular cross section, but the stick 10 can be formed in any suitable cross sectional shape, including but not limited to rectangular and triangular.

It is desired that the stick 10 take about thirty days to sufficiently diminish in size (by dissolving) to pass beyond the trap 18, thereby providing a month's worth of freshening action. The rate of dissolution will vary, of course, with the stick dimensions and the type of chemical used to form the stick 10. It is anticipated that a binder will be required for the stick to retain its shape until dissolved.

The compounds used to form the stick can include a deodorizer, a sanitizer, an antimicrobial compound and a fragrance. The stick 10 can also be unscented.

It is understood that the embodiments of the invention described above are only particular examples which serve to illustrate the principles of the invention. Modifications and alternative embodiments of the invention are contemplated which do not depart from the scope of the invention as defined by the foregoing teachings and appended claims. It is intended that the claims cover all such modifications and alternative embodiments that fall within their scope.

I claim as my invention:

1. A solid chemical stick for treating a sink drain pipe, the sink comprising a basin and a drain guard installed in the basin, the drain pipe being connected to the drain guard and having a U-shaped trap, the solid chemical stick having dimensions that enable the stick to fit through the drain guard but that prevent the stick from moving beyond the U-shaped trap.

2. The solid chemical stick of claim 1 wherein the stick is cylindrical.

3. The solid chemical stick of claim 2 wherein the stick has a substantially circular cross section and a length of about at least four and one half inches.

4. The solid chemical stick of claim 3 wherein the stick is made from a solid, dissolvable chemical.

5. The solid chemical stick of claim 4 wherein the stick diminishes in size as it dissolves until it can be swept away by drain water passing through the drain.
